(19) United States
(12) Plant Patent Application Publication
(10) Pub. No.: US 2009/0217428 P1
(43) Pub. Date: Aug. 27, 2009

(54) Actinidia chinensis plant named 'W45'

(50) ;

(76) Inventor: Donald Skelton, Rangiriri (NZ)

Correspondence Address: HortisUSA, PO Box 3109, Camarillo, CA, 93011, US

(21) Appl. No.: 12/072,480

(22) Filed: Feb. 26, 2008

(51) Int. Cl. A01H 5/00

(52) U.S. Cl. PLT/156

(57) ABSTRACT

A new and distinct Actinidia chinensis cultivar named ‘W45’ is disclosed, characterized by having distinctive yellow/green flesh fruit and a season harvest time of Mid-April. Additionally, the new variety produces fruit with very soft, slight pubescence and with a distinctive blunt stylar end shape and square shoulders. The new variety is suitable for commercial production of kiwi fruit.

### LATIN NAME OF THE GENUS AND SPECIES

Actinidia chinensis

### VARIETY DENOMINATION

‘W45’

### BACKGROUND OF THE INVENTION

The new cultivar is a product of a planned breeding program, begun in 1975 under the direction of the inventor, Donald Alfred Skelton, a citizen of New Zealand. The seed parent is the unpatented, proprietary seedling variety referred to as Actinidia chinensis ‘R55’ The pollen parent is the unpatented, proprietary seedling variety referred to as Actinidia chinensis ‘CMW85 .’

Fruit of the new variety was first evaluated in 2001 with favorable results. After the first evaluation, the new variety was grafted onto 5 seedling rootstocks; 3 of A. chinensis and 2 of A. deliciosa. Subsequent evaluations of the variety have shown the characteristics to be true to type.

### SUMMARY OF THE INVENTION

The cultivar ‘W45’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘W45’ These characteristics in combination distinguish ‘W45’ as a new and distinct Actinidia chinensis cultivar:
1. Distinctive yellow/green flesh fruit
2. Seasonal harvest time of mid April
3. Very soft, downy pubescence on fruit, with weak adherence to the fruit skin.
4. Widely obovate fruit shape.
5. Fruit with a distinctive blunt stylar end shape and square shoulders.

### COMPARISON TO PARENT

Plants of the new cultivar ‘W45’ are similar to plants of the seed parent, Actinidia chinensis ‘R55’ in most horticultural characteristics, however, plants of the new cultivar ‘W45’ produce larger size fruit, of a more greenish hue. Additionally, the new variety has new foliage emerging typically six weeks earlier than ‘R55.’

### COMMERCIAL COMPARISON

The new variety is best compared to the commercial variety, ‘Hort16A,’ U.S. Plant Pat. No. 11,066. ‘W45’ is similar to ‘Hort16A’ in many horticultural characteristics, however, ‘W45’ produces fruit with yellow/green flesh coloration whereas ‘Hort16A’ produces fruit of a golden yellow. Additionally, the new variety produces a widely obovate shaped fruit, compared to ovoid. Fruit of the new variety ‘W45’ are distinctively blunt on the stylar end, compared to the strong pointed stylar end shape of ‘Hort16A.’ Fruit shape differences can also be noted in the shoulder shape. Whereas ‘W45’ produces fruit with strongly squared shoulder, ‘Hort16A’ fruit have rounded shoulders.

### BRIEF DESCRIPTION OF THE PHOTOGRAPH

The accompanying photograph in FIG. 1 illustrates in full color typical foliage and flowers on plants of ‘W45.’

FIG. 2 illustrates in full color typical flowers of ‘W45.’

FIG. 3 shows examples of typical fruit harvested from ‘W45.’

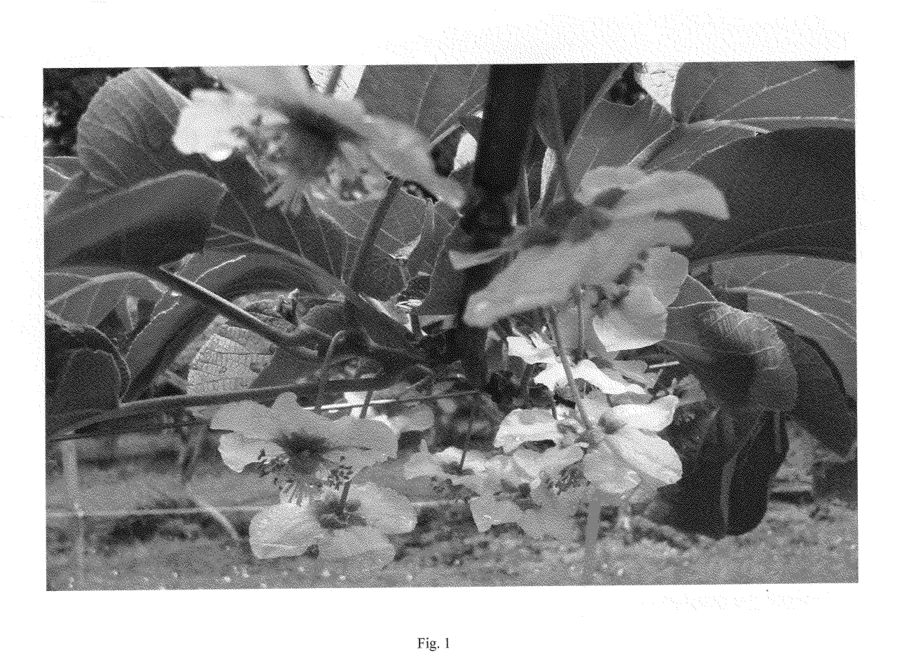

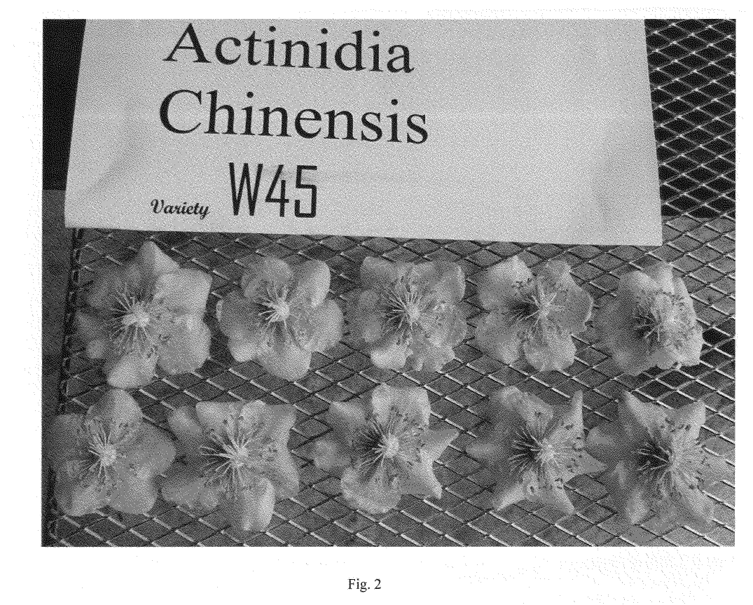

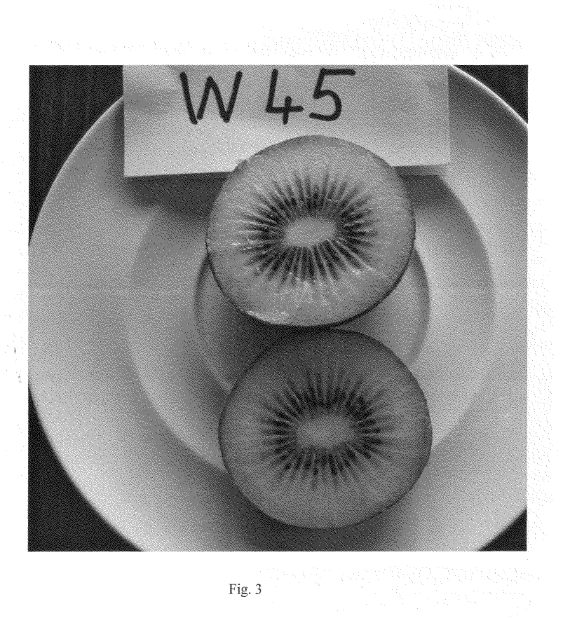

The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart except where general terms of ordinary dictionary significance are used. The following observations and measurements describe ‘W45’ plants grown under outdoor commercial trial conditions in Rangiriri New Zealand. The growing temperature ranged from 10° C. to 25° C. with no precipitation. Measurements and numerical values represent averages of typical plant types.
Botanical classification: Actinidia chinensis cultivar ‘W45.’
Propagation: W45 can be successfully grafted onto rootstocks of Actinidia chinensis or Actinidia deliciosa.
Plant:
Sex expression: Female
Ploidy: Female
Foliage:
Leaf:
   Average length.—Range between 16-18.5 cm.
   Average width.—Range between 19-23 cm.
   Shape of blade.—Broadly ovate.
   Apex.—Rounded.
   Base.—Cordate, not overlapping.
   Attachment.—Petioled.
   Margin.—Ciliate.
   Texture of top surface.—Very lightly puckered.
   Texture of under side.—Moderately glossy, non-pubescent.
   Color.—Mature foliage upper side: Near RHS Green 137A. Mature foliage under side: Near RHS Green 141B.
   Petiole.—Length. Range between 8-18 cm. Pubescence: None. Other characteristics: Moderate anthocyanin on upperside of petiole.
Flower:
   Bud break.—Mid August.
   First flower.—Early October.
   Diameter.—Approximately 5 to 7 cm.
   Petal quantity.—5 or 7 per flower.
   Petals overlapping.—Yes.
   Primary color.—white.
   Filament color.—white.
   Anther color.—brown.
   Attitude of styles.—semi-erect.
   Hair on ovary.—dense.
   Color of ovary.—white.
   Number of sepals.—Approximately 5 to 7.
   Color of sepals.—RHS Yellow-Green 144C.
Fruit:
   Color outer pericarp.—Near RHS Yellow-Green 145B.
   Color inner pericarp.—Near RHS Yellow-Green 145B.
   Core color.—Near RHS White 155C.
   Brix at consumption.—16.5.
   Brix at harvest.—7.00%.
   Average weight.—124 grams.
   Minimum weight.—104 grams.
   Maximum weight.—135 grams.
   Length.—Avg. 65 mm.
   Width.—Avg. 57 mm.
   Core diameter (maximum).—18 mm.
   Core diameter (minimum).—10 mm.
   Locule number.—30.
   Fruit peduncle length.—51 mm.
   Fruit peduncle width.—3.1 mm.
   General shape.—broad ovoid.
   Median cross section.—circular.
   Stylar end shape.—slightly blunt.
   Shoulder shape.—squared.
   Calyx ring.—present.
   Calyx ring expression.—strong.
   Skin color at harvest.—Near RHS Yellow-Green 144A.
   Hair on fruit skin.—downy.
   Hair adherence to skin.—weak.
   Fruit core shape.—transversely eliptic.
   Core-woody spike.—medium.
   Mature seed color.—black.
   Dried seed.—brown.
   Harvest time.—mid April.
   Overall cropping quantity.—heavy.
Other characteristics:
Storage life: Storage life is a minimum of 3 months at 2° C.
Disease/pest resistance: Neither resistance nor susceptibility to pathogens and pests common to Actinidia chinensis have been observed.

What is claimed is:

1. A new and distinct cultivar of Actinidia chinensis plant named ‘W45’ as herein illustrated and described.
